(19) United States
(12) Patent Application Publication
(10) Pub. No.: US 2020/0222855 A1
(43) Pub. Date: Jul. 16, 2020

(54) PROCESSES OF PRODUCING BIODIESEL AND BIODIESEL PRODUCED THEREFROM

(71) Applicant: Archer Daniels Midland Company, Decatur, IL (US)

(72) Inventors: Paul D. Bloom, Forsyth, IL (US); John Inmok Lee, Decatur, IL (US)

(73) Assignee: Archer Daniels Midland Company, Decatur, IL (US)

(21) Appl. No.: 16/637,436

(22) PCT Filed: Aug. 7, 2018
(86) PCT No.: PCT/US18/45515

§ 371 Date: Feb. 7, 2020

Related U.S. Application Data
(60) Provisional application No. 62/543,625, filed on Aug. 10, 2017.

(51) Int. Cl. B01D 61/02; B01D 11/04; C07C 67/03; B01D 61/04; C07C 67/48

(52) U.S. Cl. CPC B01D 61/027; B01D 11/0415; B01D 17/02; B01D 61/04; C07C 67/48; C07C 67/03

(57) ABSTRACT

The present disclosure discloses processes for treating, producing, or producing and treating biodiesel. Products produced with the various processes of the present invention are also disclosed.

### TECHNICAL FIELD

The present disclosure relates generally to biodiesel and processes for producing biodiesel. More particularly, the present disclosure relates to processes for improving the quality of biodiesel fuel by removing impurities and contaminants, such as sterol glycosides and other unsaponifiables.

### BACKGROUND ART

Biodiesel is used as an additive to petroleum-derived diesel fuel or as a substitute for petroleum-derived diesel fuel in diesel (compression-ignition) engines, and is comprised of the ethyl or methyl esters of fatty acids of biological origin. Starting materials for the production of biodiesel include, but are not limited to, materials containing fatty acids. These materials include, without limitation, triacylglycerols, diacylglycerols, monoacylglycerols, phospholipids, esters, free fatty acids or any combinations thereof.

The fatty acids used to produce the biodiesel may originate from a wide variety of natural sources including, but not limited to, vegetable oil, canola oil, safflower oil, sunflower oil, nasturtium seed oil, mustard seed oil, olive oil, sesame oil, soybean oil, corn oil, peanut oil, cottonseed oil, rice bran oil, babassu nut oil, castor oil, palm oil, palm oil, rapeseed oil, low erucic acid rapeseed oil, palm kernel oil, lupin oil, jatropha oil, coconut oil, flaxseed oil, evening primrose oil, jojoba oil, camelina oil, tallow, beef tallow, butter, chicken fat, lard, dairy butterfat, shea butter, biodiesel, used frying oil, oil miscella, used cooking oil, yellow trap grease, hydrogenated oils, derivatives of the oils, fractions of the oils, conjugated derivatives of the oils, and mixtures of any thereof.

Some components of biodiesel such as, for example, the esters of saturated fatty acids, may cause the development of crystals when the biodiesel is subjected to cold conditions. These crystallized materials interrupt fuel flow, and must be addressed by the application of heat—for example, towing the affected vehicle to a warm garage or applying a heat source such as a heat blower to the fuel lines and systems of the vehicle. Under these conditions, fuel flow is restored when the crystallized material melts, and the esters of saturated fatty acids can pass into the combustion chamber for burning.

Fouling of filters and injector systems by precipitates in biodiesel is another significant problem. One class of precipitating impurity that can occur in biodiesel is the steryl glucosides. Even low concentrations of steryl glucosides may foul fuel filters, injector bodies and injector nozzles. Unfortunately, the melting point of the steryl glucoside precipitates is very high (on the order of 240 degrees Celsius), so that unlike the esters of saturated fatty acids, once the precipitates have formed they cannot be easily removed from fouled filters and other surfaces by melting. Consequently, steryl glucosides may accumulate and form a refractory gum-like material requiring disassembly and cleaning of the injectors, thus increasing the operating expense of the diesel engine.

Cleaning of components fouled with steryl glucosides is still further complicated by the insolubility of steryl glucosides in most solvents. The steryl glucosides are soluble in pyridine, dioxane and dimethylformamide, but these are not found in the usual diesel repair facility, and their health hazards make these solvents unsafe for use outside of a fume hood. Consequently, the build-up of the steryl glucosides on diesel engine components usually requires labor-intensive abrasive cleaning or an expensive replacement of fuel injectors and other fouled components.

Moreover, steryl glucosides can precipitate to form an amorphous cloud-like substance and increase the filter blocking tendency of biodiesel even at temperatures well above those that are associated with the crystallization of the esters of saturated fatty acids. Even low levels of the steryl glucosides (i.e., 10-90 ppm) in the biodiesel can form aggregates with fatty acid methyl esters and accelerate filter plugging. And at cold temperatures, the cold-flow problems caused by alkyl esters of saturated fatty acids such as triacylglycerols, diacylglycerols and monoacylglycerols may certainly be compounded by the presence of the steryl glycosides.

The biodiesel industry has addressed this compound problem from the presence of steryl glucosides alongside those materials which tend to crystallize at lower ambient temperatures, such as but not being limited to the esters of saturated fatty acids, by introducing a cumbersome and expensive “cold filtration step” in the processing of biodiesel.

In a typical biodiesel manufacturing process, such as the well-known Connemann process (U.S. Pat. No. 5,354,878, “Process for Continuous Production of Lower Alkyl Esters of Higher Fatty Acids”), oil, methanol and catalyst enter a reactor near the top. The oil is converted to biodiesel, releasing glycerol from the oil, and a heavy phase containing glycerol separates and falls to the bottom. The biodiesel phase, which contains methanol, is recovered and heated in a vacuum dryer to remove residual water and recover methanol. Typically, the biodiesel can be about 120-145 degrees C. (˜250-293 degrees F.) when exiting the vacuum dryer. The biodiesel at this stage may contain steryl glucosides and other impurities, and would not likely pass a cold soak filterability and/or a cold soak filter blocking tendency test due to the presence of impurities that precipitate and cause flow problems under cold conditions.

Consequently, a cold filtration step is typically performed, in which the biodiesel exiting the stripper is chilled to less than 40 degrees C., and in many cases, to 10 degrees C. or less. The chilled biodiesel is held in tanks, sometimes for as long as for 24-36 hours, and a finely divided mineral-based powder material, such as bleaching clay or diatomaceous earth, is added as a “body feed.” In these cold conditions, impurities, primarily esters of sterol with sugars, coagulate and interact with the body feed to form filterable particles. After the formation of filterable particles, the body feed and precipitated impurities are removed with a pressure leaf filter. Leaf filters require an additional coating of filter aid. The steryl glucosides cannot be removed by filtration without the cooling/holding, and the body feed is needed to prevent “blinding” (blocking, occluding) of the leaf filters used in the filtration process.

These process steps are expensive and cumbersome, but nevertheless are commonly used for lack of a better alternative. These steps add significant energy costs to chill the biodiesel and significant costs for the filter aid, such as bleaching earth, which must also be disposed of, adding yet more expense. In addition, the incubation step requires significant investments in insulated holding tanks. Further, the filter aids tend to retain biodiesel, resulting in yield losses which add even more expense in the form of lost biodiesel and lost tax credits, such as Blenders Credit or Producer's Credit.

Filtration has required the use of particulate filter aids which are insoluble in biodiesel. Mineral-based filter aids have been employed (U.S. Pat. No. 7,635,398, J. Amer. Oil Chem. Soc. 87(3) 337-345 2010), which on the industrial scale requires elaborate filter mechanisms to remove the particulate filter aids, as filter aid impurities remaining in biodiesel cause problems. United States Patent Application Publication No. US2006260184 and US2007151146 also teach contacting biodiesel with filter aids and filtration.

Expensive chilling of biodiesel to less than 38 degrees C. is required by the cold filtration processes of U.S. Pat. No. 9,109,170. The step of chilling biodiesel after leaving the vacuum dryer is energy intensive; as many as three heat reducing stages may be needed to chill biodiesel. The heat exchangers required for cooling foul during use, and require regular cleaning. In addition, the process requires the addition of filter aids which require special filters, such as leaf filters, for removal of the filter aids.

U.S. Pat. No. 8,647,396 teaches a cumbersome and impractical washing step in which biodiesel must be washed with water. The solubility of water in biodiesel is sufficiently high that water is a contaminating impurity, and expensive steps were needed to remove the water, which was present at about 1000 ppm. The American Society for Testing Materials (ASTM) has developed standards for biodiesel fuels, with the most common being D-6751. ASTM D-6751 sets commercial quality specifications required for biodiesel fuel. The D-6751 standard requires water and sediment to be less than 0.050 percent, by volume, as measured by ASTM D-2709.

Patent Cooperation Treaty Publication No. WO2010107446 describes a method of removing impurities from biodiesel by chilling and storage at reduced temperatures for a period of time, then passage through an ion exchange resin. Chilling the large volumes of biodiesel is expensive and time-consuming; furthermore, the accumulation of precipitated impurities on the ion-exchange resin would necessitate frequent back-washing of the resin, reducing productivity.

Patent Cooperation Treaty Publication No. WO2012099523 describes an improved self-cleaning filter assembly that does not require a filter aid; however, the biodiesel is expensively first heated to 60 degrees C., then chilled to 15 degrees C. to form precipitates, and the use of additives to improve filterability is recommended.

Enzyme-based hydrolysis of steryl glucosides has been described (Patent Cooperation Treaty Publication WO2012099523); this process is unattractive for the following reasons: the expense of the enzyme, contact between biodiesel and the water required for hydrolysis, and the necessity of removing the water and enzyme after the process.

Distillation of biodiesel to remove impurities, including steryl glucosides, is taught in United States Patent Application No. US2008282606. Although the distillation of the biodiesel may produce a biodiesel having an acceptable filter blocking tendency (FBT) value or that may pass a modified ASTM 6217 test, the distillation procedure is not economically acceptable. Distillation requires expensive, often dedicated, equipment, and undesirably exposes the biodiesel to heat, which promotes oxidative damage of double bonds in the biodiesel and reduced storage stability of the distilled biodiesel.

Biodiesel has unusual solvent properties, leading to significant incompatibility with many materials. Thus, removal of impurities from biodiesel by simple membrane filtration has been impractical because most membrane materials swell or are rapidly dissolved by the biodiesel.

United States Patent Application No. US2008092435 relies on microfiltration to purify biodiesel, using filter membranes specifically made from hydrophilic or slightly hydrophilic materials. They take pains to point out the “Hydrophobic materials are not the preferred type of membrane” at [0027]. However, the membrane is chemically unstable in contact with biodiesel, causing loss of membrane flux and decreased performance, i.e. loss of rejection of unwanted contaminants. To overcome this loss of flux, high operating pressures are necessary for the separation. Further, impurities and contaminants containing hydroxyl groups, such as sterol glucosides, methanol, water, and glycerin are poorly separated and are transported through the membranes with biodiesel.

Limits on water, sulfur and phosphorus in the USA are listed in ASTM D-6751. Limits on water, sulfur, phosphorus, acid value (affected by free fatty acids), monoglycerides, diglycerides, and triglycerides in Europe are listed in EN 14214. The clarity and color of biodiesel are not generally subject to quality requirements or specifications; we have by the present invention developed means for improving both of the color and clarity of biodiesel, in particular, through the provision of improvements in the processing of biodiesel to remove steryl glucosides and other impurities.

### SUMMARY OF THE INVENTION

We have developed a process for removing impurities, including steryl glucosides, from biodiesel that may optionally use, but importantly does not require, the cold filtration steps of chilling, adding filter aid, and filtering through leaf filters. Steryl glycosides and other impurities are removed by placing the biodiesel in contact with a non-polar, hydrophobic, and chemically stable organic solvent nanofiltration (OSN) membrane capable of removing steryl glycosides and other impurities in biodiesel from the biodiesel. After removing the impurities from the biodiesel, the biodiesel has improved characteristics.

In an embodiment, the present disclosure provides a process for producing biodiesel with a reduced steryl glycoside content, comprising placing biodiesel in contact with an organic solvent nanofiltration membrane capable of removing steryl glycosides from the biodiesel, wherein the biodiesel passes through the membrane.

In another embodiment, after contact with the membrane, the steryl glycoside content is reduced to an extent whereby the biodiesel passes at least one of the ASTM D7501 cold soak filterability test or the Canadian standard method CGSB-3.0 No. 142.0 cold soak filter blocking tendency test.

In this regard, ASTM D7501 will be understood as involving the determination of the filtration time (in seconds) that is required for 300 mL of a biodiesel to be filtered through a single 0.7 micrometer glass fiber filter under a controlled vacuum of from 70 to 85 kPa (21 to 25 inches Hg), after the 300 mL of biodiesel has been stored at from 4 to 5 degrees Celsius (39 to 41 degrees Fahrenheit) for 16 hours and then allowed to warm to a temperature of from 24 to 26 degrees Celsius (75 to 79 deg. F.). A passing filtration time for ASTM D7501 is 360 seconds or less.

The CGSB-3.0 No. 142.0 cold soak filter blocking tendency test measures the relative filterability of biodiesels after a cold soak cycle as a result of the propensity of minor components of some biodiesel esters, for example, in the form of saturated monoglycerides, to separate from a blend of biodiesel and isoparaffinic solvent above the cloud point of a biodiesel fuel blend. In this particular test, a sample of biodiesel is first conditioned to erase its thermal history. A blend of 20 percent by volume of the biodiesel sample in an isoparaffinic solvent is prepared and “cold soaked” at 1 degree Celsius for 16 hours. The sample is then warmed to 25 degrees Celsius for from 2 to 4 hours. After warming, the sample is then passed at a constant 20 mL/minute through a 1.6 micrometer glass fiber filter medium. The pressure drop across the filter is monitored until 300 mL of the blend has passed through the filter, or if a maximum pressure drop of 105 kPa is reached before this time, the actual volume filtered at the time the maximum pressure drop has been reached is recorded and used to calculate the cold soak filter blocking tendency result. Results of the CSFBT test can range from 1 for a biodiesel with very good filterability to more than 10 for a fuel with poor filterability. A passing filtration value would be no greater than 1.8.

In an alternative embodiment, after contact with the OSN membrane, the steryl glycoside content is reduced to an extent whereby the biodiesel after passing through the membrane has an ASTM D7501 cold soak filterability test time of less than 90 seconds.

In a yet further embodiment, the temperature of the biodiesel while being placed in contact with the organic solvent nanofiltration membrane is in the range of from 40-120 degrees C.

In another embodiment, a process is provided for concurrently removing one or more other impurities selected from the group consisting of water, phosphorus, sulfur, free fatty acids, monoglycerides, diglycerides, and triglycerides from a biodiesel, by placing the biodiesel in contact with a non-polar, hydrophobic, and chemically stable organic solvent nanofiltration (OSN) membrane capable of removing steryl glycosides and the one or more other impurities from the biodiesel.

In an embodiment, the amount of any one of phosphorus, sulfur, diglycerides or monoglycerides in the biodiesel after being placed in contact with the membrane is less than half the amount of the impurity in the biodiesel before being placed in contact with the membrane.

In yet a further embodiment, a process is provided for reducing at least one of the Lovibond red value or the Lovibond yellow value of the biodiesel, by contacting a biodiesel with such an OSN membrane.

In another embodiment, after being placed in contact with the membrane the Lovibond red value of the biodiesel is not greater than 2.0 or the Lovibond yellow value of the biodiesel is not greater than 35, as determined in a one inch cell.

In an alternative embodiment, the biodiesel comprises fatty acids derived from the group consisting of vegetable oil, canola oil, safflower oil, sunflower oil, nasturtium seed oil, mustard seed oil, olive oil, sesame oil, soybean oil, corn oil, peanut oil, cottonseed oil, rice bran oil, babassu nut oil, castor oil, palm oil, palm kernel oil, rapeseed oil, low erucic acid rapeseed oil, palm kernel oil, lupin oil, jatropha oil, coconut oil, flaxseed oil, evening primrose oil, jojoba oil, tallow, beef tallow, butter, chicken fat, lard, dairy butterfat, shea butter, biodiesel, used frying oil, oil miscella, used cooking oil, yellow trap grease, hydrogenated oils, gums, soapstock, acid oil, derivatives of the oils, fractions of the oils, conjugated derivatives of the oils, and mixtures of any thereof.

In yet another embodiment, the present disclosure encompasses a composition produced by placing biodiesel in contact with an organic solvent nanofiltration membrane capable of removing steryl glycosides from the biodiesel, wherein the biodiesel passes through the membrane, wherein the composition has a detectable spectrophotometric transmittance.

In a further embodiment, the spectrophotometric transmittance of the biodiesel after being placed in contact with the membrane is greater than 20%.

In yet another embodiment, the present disclosure encompasses a composition produced by the embodiments listed herein.

### DETAILED DESCRIPTION OF EMBODIMENTS

One method for predicting the behavior of biodiesel under cold conditions is to determine the “Cold Soak Filterability,” as indicated by the ASTM D7501 method, as summarized above.

An alternative method of testing biodiesel is the Canadian Standard Method C**/CGSB-3.0 No. 142.0, “Cold Soak Filter Blocking Tendency of Biodiesel (B100)”, also as summarized above. As previously noted. minor components of some biodiesel esters, including saturated monoglycerides and steryl glycosides, can separate above the cloud point of a biodiesel fuel blend. The CSFBT test assesses the propensity of these materials to separate from a blend of biodiesel and isoparaffinic solvent after a cold soak cycle, yielding a dimensionless value indicative of the relative tendency of a given biodiesel fuel to plug or block a filter, based on the relative pressure increase across a filter observed in filtering different biodiesels or on the volumes filtered of various biodiesels once a limiting pressure drop has been reached.

What is meant by “chilling” is the process of reducing the temperature of biodiesel to below ambient. What is meant by “unchilled” is that the biodiesel has not been subjected to the usual industrial practice of chilling to 10 degrees C. and holding to allow precipitates to form.

We have developed methods using a so-called “organic solvent nanofiltration” membrane to remove steryl glycosides from unchilled, unfiltered biodiesel, that is, directly after the vacuum stripper. This enables the avoidance of the chilling, holding, addition of filter aid, the use of leaf filters, the loss of biodiesel trapped in the spent filter aid, and the expense of disposal of the filter aid. In addition, we developed methods to remove water, phosphorus, sulfur, free fatty acids, monoglycerides, diglycerides, and triglycerides as well as substantially clarify the biodiesel using an OSN membrane, as well as improve the Cold Soak Filter Blocking Tendency and lower the Filter Blocking Tendency of the biodiesel. The improved biodiesel has a decreased amount of steryl glycosides and may be of greater clarity (higher in transmittance) and may be lower in one or more of water, phosphorus, sulfur, free fatty acids, chlorophyll and other color-imparting impurities, monoglycerides, diglycerides, and/or triglycerides than untreated biodiesel.

Petroleum diesel fuel often must undergo a desulfurization process to reduce the content of sulfur. This is carried out by hydrogenation or hydrodeoxygenation, requiring expensive equipment and high temperatures. We have developed a method using an organic solvent nanofiltration membrane for the desulfurization of diesel fuel; the method obviates the expensive equipment and high temperatures normally needed for petroleum diesel fuel desulfurization.

The invention is further explained by use of the following illustrative examples.

### Example 1

A flat sheet non-polar organic solvent nanofiltration (OSN) membrane (PMS-600 PuraMem™ membrane, Evonik Industries, Chicago, Ill., USA, molecular weight cut off nominally 600, membrane area 0.0062 m²) was secured into a flat sheet membrane holder. Toluene was passed through the membrane to remove the preservative, then the toluene was flushed out with biodiesel.

Unfiltered canola biodiesel (fatty acid methyl esters, Archer Daniels Midland Co., Velva, N. Dak., USA) was obtained. The biodiesel had been subjected to vacuum stripping at 120 degrees C., but had not been treated by the usual chilling to 10 degrees C. and filtering with filter aid in a leaf filter as carried out in commercial biodiesel production to reduce the content of steryl glycosides in the biodiesel. A closed system was pressurized with nitrogen (20 bar) on the upper side of the membrane to provide cross-membrane driving force. Unchilled, unfiltered biodiesel at 22 degrees C. containing 55.80 mg/kg steryl glycosides (measured by gas chromatography after derivatization) was purified by passing the biodiesel through the membrane. In 1.5 hours, 50 mL of purified biodiesel permeate containing 5.25 mg/kg steryl glycosides was obtained. Thus, a 91% reduction of steryl glycosides was obtained without chilling the biodiesel to below ambient temperature and without contacting the biodiesel with filter aid.

### Example 2

Unchilled, unfiltered canola biodiesel (438.50 grams, ADM, Velva, N. Dak., USA) containing 32.15 mg/kg steryl glycosides was hydrated by adding water (1.05 grams) and stirred for 30 minutes, yielding biodiesel containing 0.3% water. This was passed through the organic solvent nanofiltration membrane substantially according to Example 1 except the pressure was raised to 27 bar to yield 100 grams of purified diesel. After the addition of water, the level of steryl glycosides in the OSN filtered biodiesel was below detection limits (<2 mg/kg), without chilling the biodiesel to below ambient temperature and without contacting the biodiesel with filter aid. Thus instead of chilling and filtering, steryl glycoside removal was carried out by simply adding a trace of water and passing the unfiltered biodiesel through an OSN filtration membrane.

### Example 3

Unchilled, unfiltered canola biodiesel (ADM, Velva, N. Dak., USA) containing 19.64 mg/kg sterol glycosides, 0.34% monoacylglycerols and 0.09% diacylglycerols was passed through a flat sheet organic solvent nanofiltration membrane (PMF flux, Evonik Industries) having a nominal molecular weight cutoff of 500 in substantially the same arrangement as in Example 1 except the pressure applied was 26 bar. Permeate (35 ml) was collected in 1.5 hours and tested (Table 1).

TABLE 1

| | | | Rejection by |
|---|---|---|---|
| | Feed | Permeate | membrane (%) |
| Sterol glycosides | 19.64 mg/kg | 0 mg/kg | 100 |
| Monoacylglycerols | 0.34% | 0.27% | 21 |
| Diacylglycerols | 0.09% | 0.04% | 56 |

The Evonik PMF flux membrane was able to produce biodiesel with undetectable sterol glycoside levels while substantially reducing the content of monoacylglycerols and diacylglycerols without chilling the biodiesel to below ambient temperature and without contacting the biodiesel with filter aid.

### Example 4

Unchilled, unfiltered canola biodiesel (ADM, Velva, N. Dak., USA) containing 5.78 mg/kg sterol glycosides, 0.34% monoacylglycerols and 0.11% diacylglycerols was hydrated by adding 2.05 grams of water to 345.92 grams of biodiesel to produce biodiesel containing 0.59% water. This was passed through the flat sheet PMF flux membrane substantially as described in Example 3. Permeate (35 ml) was collected in 1.5 hours and tested (Table 2).

TABLE 2

| | | | Rejection by |
|---|---|---|---|
| | Feed | Permeate | membrane (%) |
| Sterol glycosides | 5.78 mg/kg | 0 mg/kg | 100 |
| Monoacylglycerols | 0.34% | 0.28% | 18 |
| Diacylglycerols | 0.11% | 0.03% | 73 |

The flat sheet organic solvent nanofiltration membrane was very effective at removing steryl glycosides and diacylglycerols from hydrated biodiesel, and was able to reduce the content of monoglycerides without chilling the biodiesel to below ambient temperature and without contacting the biodiesel with filter aid.

### Example 5

Unchilled, unfiltered biodiesel was placed into an agitated feed vessel, circulated through a heater set at 45 degrees C. and passed through a spiral wound OSN membrane (Evonik PuraMem™ Flux membrane, surface area: 0.12 square meters, one inch diameter) at 20.7 bar (300 psi) pressure. The flux was 15.8-17.4 liters/meter²/hour. Biodiesel passed through the membrane as permeate and steryl glycosides were retained. The retentate, containing biodiesel enriched in steryl glycosides, was recirculated to the feed vessel so that the concentration of steryl glycosides in the biodiesel feed increased. The level of steryl glycosides in the feed and the purified permeate was determined at three times as the concentration of steryl glycosides increased (Table 3).

TABLE 3
Steryl glycosides in unchilled, unfiltered canola
biodiesel as recirculation of feed was carried out.

| | | | Rejection by |
|---|---|---|---|
| Lot | Feed (mg/kg) | Permeate (mg/kg) | membrane (%) |
| 1 | 47.39 | 3.54 | 93.53% |
| 2 | 53.95 | 4.61 | 91.46% |
| 3 | 64.30 | 5.01 | 92.21% |

Even as the concentration of steryl glycosides increased in the feed, the rejection of steryl glycosides by the membrane remained high. The content of steryl glycosides in the unchilled, unfiltered biodiesel was decreased by greater than 90% after permeating through the spiral wound membrane.

### Example 6

The removal of additional impurities from unchilled, unfiltered biodiesel was tested. Biodiesel was passed through the spiral wound membrane substantially as in Example 5 and the permeate, treated biodiesel tested for impurities. The results are shown in Table 4.

TABLE 4
Removal of impurities from biodiesel after
permeating through the membrane.

| | | Concentration | |
|---|---|---|---|
| | Concentration in | in treated | Rejection by |
| Component | raw biodiesel | biodiesel | OSN (%) |
| Phosphorus (mg/kg) | 1.05 | 0.44 | 57.9 |
| Sulfur (mg/kg) | 1.71 | 0.87 | 49.2 |
| Free fatty acids (%) | 0.18 | 0.13 | 26.5 |
| Monoglycerides (%) | 0.48 | 0.32 | 34.1 |
| Diglycerides (%) | 0.28 | 0.06 | 78.7 |
| Triglycerides (%) | 1.40 | 0.15 | 89.2 |
| Chlorophyll (ppm) | 3.407 | 0.008 | 99.8 |

Significant removal of all measured impurities was achieved, with about half or more of the phosphorus, sulfur, diglycerides, and triglycerides being removed from the unchilled, unfiltered biodiesel.

### Example 7

The progress in the concentration of biodiesel after permeating through the membrane was determined. The unchilled, unfiltered biodiesel from Example 6 was heated to 45 degrees C. and passed through the spiral wound Evonik PuraMem™ Flux OSN membrane at 20.68 bar pressure substantially as outlined in Example 5. The biodiesel was purified as it passed through the membrane and the rate of permeate flux was measured. The retentate was recirculated back to the feed vessel. The permeate flux was measured and a factor known as the Volume Concentration Factor was calculated. In this equation, Vₒ is a constant that represents the starting volume of unchilled, unfiltered biodiesel, and V_{c} is an ever-decreasing number representing the volume of retentate remaining as the biodiesel passes into the permeate. The volume Concentration Factor was obtained by dividing Vₒ by V_{c}. The Biodiesel recovery represents the percentage of the starting volume (Vₒ) that was obtained as permeate through the spiral wound membrane (Table 5).

TABLE 5
Flux rate, Volume Concentration Factor, and Biodiesel
recovery obtained when recirculating biodiesel
through the spiral wound membrane.
Biodiesel temperature: 45 degrees C.; pressure: 20.68 bar.

| | Permeate | Volume | Biodiesel |
|---|---|---|---|
| | flux rate | concentration | recovery |
| Sample | (liter/m²-hour) | factor | (%) |
| 1 | 17.4 | 1.10 | 8.7 |
| 2 | 16.6 | 1.35 | 26.1 |
| 3 | 16.8 | 1.77 | 43.5 |
| 4 | 16.3 | 3.29 | 69.6 |
| 5 | 16.0 | 7.67 | 87.0 |
| 6 | 15.8 | 23.0 | 95.7 |

The permeate flux rate decreased only slightly during the 5-hour test. The Volume Concentration Factor of the recycling biodiesel retentate was 23.0, and 95.7% of the initial volume of biodiesel was recovered as membrane-purified biodiesel without chilling the biodiesel to below ambient temperature and without contacting the biodiesel with filter aid.

### Example 8

Two important operational characteristics of biodiesel, the Cold Soak Filterability and the Cold Soak Filter Blocking Tendency, were determined after permeating unchilled unfiltered canola biodiesel through the spiral wound OSN membrane substantially as described in Example 6. The cold soak filterability was determined according to ASTM D7501 (Table 6). The Cold Soak Filter Blocking Tendency was tested according to Canadian Standard Method CGSB-3.0 No. 142.0, “Cold Soak Filter Blocking Tendency of Biodiesel (B100)”. The biodiesel was heated to 60 degrees C. for three hours prior to the Cold Soak Filter Blocking Tendency (CSFBT) test.

TABLE 6

| Quality test | Required | OSN treated biodiesel |
|---|---|---|
| Cold soak | No more than 360 seconds | 89 seconds |
| Filterability | | |
| Cold Soak Filter | No greater than 1.41 | 1.05 |
| Blocking Tendency | | |

The biodiesel obtained by passing unchilled, unfiltered biodiesel through the OSN membrane passed both the Cold Soak Filterability test and the Cold Soak Filter Blocking Tendency test, without chilling the biodiesel to below ambient temperature and without contacting the biodiesel with filter aid.

### Example 9

Color removal from biodiesel from Example 6 was tested. Unchilled, unfiltered canola biodiesel (ADM, Velva, N. Dak., USA) was dark and hazy, and when tested in a spectrophotometer no detectable transmittance could be measured. After the unchilled, unfiltered canola biodiesel passed through the spiral wound membrane, a significant improvement in clarity was noted. The appearance of the biodiesel was bright and clear, similar to edible quality vegetable oil. The spectrophotometer test was repeated on the filtered biodiesel. The transmittance was 38.5%, exhibiting a substantial improvement in clarity of the oil.

The Lovibond Red and Lovibond Yellow values of the unchilled, unfiltered biodiesel were determined in a Lovibond colorimeter using a one inch Lovibond cell according to method AOCS Method Cc 13b-45. Before passing through the OSN membrane, the Lovibond Red value was 3.1 and the Lovibond Yellow value was 70. After passing through the OSN membrane, the Lovibond Red value was 0.5 and the Lovibond Yellow value was 13, illustrating the significant decrease in the color of the biodiesel without chilling the biodiesel to below ambient temperature and without contacting the biodiesel with filter aid.

The exemplary embodiments described herein are not intended to limit the invention or the scope of the appended claims. Various combinations and modifications of the embodiments described herein may be made without departing from the scope of the present disclosure and all modifications are meant to be included within the scope of the present disclosure. For instance, the various embodiments of the biodiesel treatments described herein may be used in conjunction with other embodiments of the biodiesel processing activities described herein. Further, the biodiesel treatment activities described herein may be implemented by modifying existing biodiesel processing systems and used in conjunction with existing biodiesel processing equipment.

1. A process for treating biodiesel comprising:
placing biodiesel in contact with an organic solvent nanofiltration membrane capable of removing steryl glycosides from the biodiesel, wherein the biodiesel passes through the membrane.

2. The process of claim 1, wherein the biodiesel contains a smaller amount of steryl glycosides after being placed in contact with the membrane than before being placed in contact with the membrane.

3. The process of claim 1 or claim 2, wherein before contacting the membrane, the biodiesel has not been subjected to a processing step selected from the group consisting of allowing the temperature to decrease below 40 degrees C., chilling to below ambient temperature, contacting the biodiesel with filter aid, holding the chilled biodiesel and filtering the biodiesel through a leaf filter.

4. The process of claim 1, wherein after contact with the membrane, the biodiesel passes at least one of the ASTM D7501 cold soak filterability or the Canadian standard method CGSB-3.0 No. 142.0 cold soak filter blocking tendency test.

5. The process of claim 1, wherein after contact with the OSN membrane, the biodiesel has an ASTM D7501 cold soak filterability test time of less than 90 seconds.

6. The process of any one of claims 1-5, wherein the temperature of the biodiesel while being placed in contact with the organic solvent nanofiltration membrane is in the range of from 40 to 120 degrees C.

7. The process of claim 1, wherein, after being placed in contact with the membrane the biodiesel contains a smaller amount of one or more impurity selected from the group consisting of water, phosphorus, sulfur, free fatty acids, chlorophyll, monoglycerides, diglycerides, and triglycerides, than before being placed in contact with the membrane.

8. The process of claim 7, wherein the amount of any one of phosphorus, sulfur, diglycerides or monoglycerides in the biodiesel after being placed in contact with the membrane is less than half the amount of the impurity in the biodiesel before being placed in contact with the membrane.

9. The process of claim 1, wherein at least one of the Lovibond red value or the Lovibond yellow value of the biodiesel is lower after being placed in contact with the membrane.

10. The process of claim 9, wherein after being placed in contact with the membrane the Lovibond red value of the biodiesel is not greater than 2.0 or the Lovibond yellow value of the biodiesel is not greater than 35, as determined in a one inch Lovibond cell.

11. The process of claim 1, wherein biodiesel comprises fatty acids derived from the group consisting of vegetable oil, canola oil, safflower oil, sunflower oil, nasturtium seed oil, mustard seed oil, olive oil, sesame oil, soybean oil, corn oil, peanut oil, cottonseed oil, rice bran oil, babassu nut oil, castor oil, palm oil, palm kernel oil, rapeseed oil, low erucic acid rapeseed oil, palm kernel oil, lupin oil, jatropha oil, coconut oil, flaxseed oil, evening primrose oil, jojoba oil, tallow, beef tallow, butter, chicken fat, lard, dairy butterfat, shea butter, biodiesel, used frying oil, oil miscella, used cooking oil, yellow trap grease, hydrogenated oils, gums, soapstock, acid oil, derivatives of the oils, fractions of the oils, conjugated derivatives of the oils, and mixtures of any thereof.

12. A composition produced by the process of claim 1, wherein the composition has a detectable spectrophotometric transmittance.

13. The composition of claim 11, wherein the spectrophotometric transmittance of the biodiesel after being placed in contact with the membrane is greater than 20%.

14. A product produced by the process of any one of claims 1-11.

15. A process for producing biodiesel, comprising:
mixing a fatty acid containing material with an alcohol, thus producing a biodiesel precursor mixture;
subjecting the biodiesel precursor mixture to a condition selected from the group consisting of time, an increased temperature, an increased pressure, the presence of a catalyst, and any combination thereof, thus producing a mixture of biodiesel and methanol;
removing methanol and glycerol to obtain the biodiesel; and
passing the biodiesel through an organic solvent nanofiltration membrane at a temperature of less than 125° C.;
wherein steryl glycosides are removed from the biodiesel.
